(19) United States
(12) Patent Application Publication
(10) Pub. No.: US 2016/0030640 A1
(43) Pub. Date: Feb. 4, 2016

(54) CARBON NANOTUBES AND GRAPHENE PATCHES AND IMPLANTS FOR BIOLOGICAL TISSUE

(71) Applicant: NEW YORK SOCIETY FOR THE RUPTURED AND CRIPPLED MAINTAINING THE HOSPITAL FOR SPECIAL SURGERY, New York, NY (US)

(72) Inventors: Joshua Schroeder, Modiin (IL); Colin Nuckolls, New York, NY (US); Frank P. Cammisa, Jr., Greenwich, CT (US); Celeste Abjornson, Stockton, NJ (US); Bumjung Kim, Fort Lee, NJ (US)

(21) Appl. No.: 14/776,250

(22) PCT Filed: Mar. 14, 2014
(86) PCT No.: PCT/US14/28110

§ 371 Date: Sep. 14, 2015

Related U.S. Application Data
(60) Provisional application No. 61/789,384, filed on Mar. 15, 2013.

(51) Int. Cl. A61L 27/44; A61F 2/02

(52) U.S. Cl. CPC A61L 27/443; A61F 2/02; A61L 2430/30; A61L 2430/34; A61L 2430/20

(57) ABSTRACT

The present invention relates to a novel composition comprising carbon nanotubes and/or graphene and a supporting material that can be used as an implant, patch or construct for the treatment, repair or replacement of biological tissue, including musculoskeletal tissue, fascia tissue, dura tissue, epidermal tissue, blood vessels and arteries, and organs. The present invention is also a method of manufacturing the novel composition, and a method of using the novel composition to treat, repair or replace biological tissue.

### CROSS-REFERENCE TO PRIOR APPLICATION

The present application claims priority to U.S. patent application Ser. No. 61/789,384 filed Mar. 15, 2013, which is hereby incorporated by reference in its entirety.

### FIELD OF THE INVENTION

The present invention is in the field of implants, more specifically implants or patches made of carbon nanotubes and graphene. The invention includes the implants or patches themselves, a method of making the implants or patches, and a method of using the implants or patches to repair, replace and/or treat injured and/or defective biological tissue.

### BACKGROUND OF THE INVENTION

Lumbar disc herniation (LDH) is the painful and debilitating pathological condition for which spinal surgery is most often performed. The average age for disc herniation is 40.8 years, with ages ranging from 15-74 years (Spangfort et al. (1972)). Surgery is done most often at the level of L5/S1 (50.5%) and L4/5 (47.5%). The incidence of disc surgery is growing both in the United States and world-wide. In 2011, over 500,000 surgeries for disk herniation were performed in the United States alone. To date, the only route to ease the pain of LDH is surgery, which holds risks for nerve damage.

Two anatomically different types of lumbar disc herniation have been described with regard to a penetration of the posterior annulus and longitudinal ligament, respectively. Based on this, LDH can be classified as contained or non-contained. Contained discs, which are completely covered by outer annular fibres or posterior longitudinal ligament, are not in direct contact with epidural tissue. In contrast, non-contained discs are in direct contact with epidural tissue. This differentiation between the conditions is of importance for the type of surgical treatment.

After non-operative treatments have failed, a standard discectomy is performed, which consists of an unilateral exposure of the interlaminar window and partial flavectomy to expose the dura and nerve roots as well as the intervertebral disc. Optionally this technique can be used with magnification loops and headlights or microscopy to enhance visibility. While this approach provides very favorable results in the vast majority of cases (over 90% pain reduction in primary discectomy), the partial removal of the protruding annulus disturbs the safe constraint for the gel-like nucleus pulposus. In these cases, recurrent herniations frequently occur and require repeat surgeries. The reported rate of recurrent disc herniation after primary discectomy ranges between 5% and 11%, but can increase up to 25% in non-degenerated discs. In a study of patients who underwent primary lumbar discectomy, patients with fragments and small annular defects had a recurrency rate of 1%, patients with fragments and contained disc herniation, 10%, and patients with fragments and massive posterior annular loss, 27%. The highest recurrence rate (38%) was in patients with no fragments and contained disc herniations (Carragee et al. (2003)). In another study, it was found that minor disc degeneration represents a risk factor for the recurrence of disc herniation after discectomy, more so than herniation volume.

There are two main strategies to AF repair: one is supplementing the loss of annulus by providing mechanical stability using, for example, vicryl suture or a composite alloy device, while the other aims at regenerating functional AF tissue that will in turn integrate within the AF. Cell therapy has been proposed to increase and supplement AF, as well as nucleus pulposus self-repair using scaffolds loaded with biological factors. However, these devices have failed as they lacked the strength to hold the herniation in place, as forces of 400 N are generated when a patient bends forward. Recently, electrospun nanofibrous scaffolds seeded with mesenchymal stem cells has showed promise for annulus fibrosus repair (Nerurkar et al. (2009)).

Therefore, novel strategies towards annular repair would significantly improve the presently limited surgical outcome in patients with contained disc herniations, but otherwise minor degenerative changes, which occur mainly in relatively young patients.

Lumbar disc herniation is not the only pathological orthopedic contion in need of new treatments and therapies. Due to the aging population, the need and market for orthopedic implants is growing rapidly. Over 600,000 joint replacements are performed each year in the United States alone (Christenson et al. (2007)). Orthopedic implants today do not allow a patient to return to normal active lifestyles, and the average lifetime of implants is only 10-15 years, meaning that younger patients endure several painful and expensive surgeries (Parchi et al. (2013)). Thus, there is a need for improved implants for other musculoskeletal tissue.

There is also a need for less invasive treatment, repair and/or replacement of other biological tissue, such as epidermis and organs.

### SUMMARY OF THE INVENTION

The present invention overcomes the problems in the art by providing a novel implant or patch for treating, repairing, and/or replacing a defect and/or injury in biological tissue, more specifically musculoskeletal tissue, and most specifically annular defects, without highly invasive surgical techniques. It also provides a method to manufacture the novel implant or patch, and a method to treat, repair and/or replace a defect and/or injury in biological tissue, with the implant or patch.

Thus, one embodiment of the present invention is an implant or patch composition comprised of carbon nanotubes and/or graphene. The combination of the small size of carbon nanotubes and/or graphene with the extreme strength provides a patch that can be inserted into areas as small as spinal discs, but that are strong enough to withstand biological forces created by movement, such as sitting and standing. With regard to annulus fibrosus repair, this will prevent the recurrence of disc herniation and the bulge-out of the nucleus pulposus when the annulus fibrosus is damaged.

The implant or patch composition is also comprised of one or more materials that supports the nanotubes and/or graphene, and facilitates the transfer of the nanotubes to the defect. This material can be a coating. This material, which is referred to interchangeably as supportive material or supporting material or a carrier or carrier material or coating or coating material, can be any polymer, plastic, or composite material that is biocompatible. A preferred material for the supportive and/or coating material is any plastic that is hydrophobic. This material can include, but is not limited to, polyethylene glycol (PEG), polydimethylsiloxane (PDMS), vicryl, polyethylene (PE), poly-lactic acid (PLA), polyetheretherketone (PEEK), polyglycolic acid (PGA), and polyurethane. The supporting material can be modified to create binding terminals, such as amine-terminates, to create an adhesive interface that facilitates the attachment to the adjacent tissue surrounding the defect or injury. A further advantage of the patch or implant conferred by the carrier or supporting material is that the implant is flexible and has memory allowing it to be manipulated or folded when inserted or implanted in a small defect or injury in a subject and then open or unfold to its original shape and size upon insertion or implantation.

The implant or patch composition can also comprise a biocompatible adhesive to bind the implant or patch composition to the biological tissue or to the extracellular matrix of the tissue of the subject upon implantation. This adhesive can work by making the implant or patch composition hydrophilic or hydrophobic. The adhesive can also bind to collagen, cartilage, bone, or another component of the extracellular matrix. This adhesive includes, but is not limited to, polyethylene glycol and poly-L-lysine.

In a further preferred embodiment, the adhesive is a chemical or biological moiety that binds to a component of the extracellular matrix of the host tissue, including but not limited to, collagen, fibronectin and laminin. In a preferred embodiment, the chemical moiety is an organic reactive carbonate. In another preferred embodiment, the biological moiety is a “microbial surface component recognizing adhesive matrix molecules” or MSCRAMMs. MSCRAMMs allow bacteria or other entities to attach to an extracellular matrix component in a tissue. A most preferred MSCRAMM is encoded by the CNA gene, known as the CollageN Adhesion protein or CNA of Staph aureus. These moieties are described in co-owned U.S. Pat. No. 8,440,618, herein incorporated in its entirety.

The implant or patch composition can also comprise other agents that facilitate migration, integration, regeneration, proliferation, and growth of cells into and around the implant or patch composition, and/or the injury or defect, and/or promote healing of the injury or defect, and/or are chondrogenic and osteogenic, i.e., build bone and cartilage.

The implant or patch composition can also comprise other agents, including but not limited to, cytokines, chemokines, chemoattractants, anti-microbials, anti-virals, anti-inflammatories, pro-inflammatories, bone or cartilage regenerator molecules, and chemotherapy medication.

A further embodiment of the present invention is a novel method for producing or manufacturing the carbon nanotube and/or graphene implant or patch composition. The method for producing or manufacturing an implant or patch comprising carbon nanotubes comprises the steps of:

1. preparing a solution of carbon nanotubes for mixing;

2. mixing the carbon nanotube solution with material that supports the carbon nanotube;

3. pouring the mixture of carbon nanotube solution and supporting material onto a substrate to form a thin film; and

4. hardening the film.

A further embodiment of the present invention is a novel method for producing or manufacturing a patch comprising graphene. This method comprises the steps of:

1. preparing a large scale graphene on copper foil;

2. laminating the graphene on copper onto a material that supports the graphene to make a film;

3. removing the copper;

4. adding additional supporting material to the film of graphene and supporting material; and

5. hardening the resulting material.

This method is repeated a number of times to obtain a multilayered implant.

The resulting patch can be about 50 nm to 15 mm thick. However, one of skill in the art would recognize the size of the patch or implant will depend on its use, i.e., the biological tissue being treated, repaired and/or replaced.

The preferred supporting material in both methods is polydimethylsiloxane (PDMS).

A further embodiment of the present invention is a method of producing or manufacturing an implant or patch composition that comprises carbon nanotubes and graphene.

Yet a further embodiment of the present invention is a novel method to treat, repair and/or replace defects and/or injuries to biological tissue by implanting the carbon nanotube and/or graphene implant or patch into a subject to treat, repair and/or replace defects and/or injury to biological tissue. In a preferred embodiment, the tissue is musculoskeletal tissue, including bone, tendon, ligaments, cartilage, fascia, vascular tissue, joints, and the discs of the spine. Other biological tissue that can be treated, repaired or replaced include, but is not limited to, fascia, epidermal tissue, vascular tissue and organs. In a most preferred embodiment the implant is used to treat, repair, and/or replace the annulus fibrosus tissue of the discs of the spine. Moreover, the implants of the present invention are suitable for implantation into mammals, more specifically humans.

### BRIEF DESCRIPTION OF THE DRAWINGS

For the purpose of illustrating the invention, there are depicted in drawings certain embodiments of the invention. However, the invention is not limited to the precise arrangements and instrumentalities of the embodiments depicted in the drawings.

FIG. 1 is a schematic drawing of the manufacturing method for patches or implants comprising carbon nanotubes.

FIG. 2 is a schematic drawing of the manufacturing method for patches or implants that comprise graphene.

FIG. 3 is a schematic drawing of an implant.

### DETAILED DESCRIPTION OF THE INVENTION

The present invention is a novel composition for treating, repairing and/or replacing biological tissue comprising carbon nanotubes and/or graphene, and a carrier or supporting material. The composition can further comprise additional agents, including but not limited to, adhesives, cytokines, chemokines, chemoattractants, anti-viral agents, anti-microbial agents, anti-inflammatories, pro-inflammatories, bone or cartilage regenerator molecules, and chemotherapy medication.

The present invention is also a novel method of manufacturing such a composition, and the use of the composition to treat, repair and/or replace a defect and/or injury in biological tissue in a subject in need thereof.

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The terms “implant”, “patch” and “construct”, are used interchangeably throughout this application and means any material inserted or grafted into the body that maintains support and tissue contour.

The term “subject” as used in this application means an animal with an immune system such as avians and mammals. Mammals include canines, felines, rodents, bovine, equines, porcines, ovines, and primates. Avians include, but are not limited to, fowls, songbirds, and raptors. Thus, the invention can be used in veterinary medicine, e.g., to treat companion animals, farm animals, laboratory animals in zoological parks, and animals in the wild. The invention is particularly desirable for human medical applications

The term “in need thereof” would be a subject known or suspected of having an injury to or defect in any biological tissue including, but not limited to musculoskeletal tissues including, but not limited to cartilage, bone, tendon, ligaments, and the discs of the spine, and fascia tissue, dura tissue, epidermal tissue, arteries and blood vessels, and organs.

The terms “treat”, “treatment”, and the like refer to a means to slow down, relieve, ameliorate or alleviate at least one of the symptoms of the defect or injury or reverse the defect or injury after its onset.

The term “repair” and the like refer to any correction, reinforcement, reconditioning, remedy, making up for, making sound, renewal, mending, patching, or the like that restores function. Accordingly, the term “repair” can also mean to correct, to reinforce, to recondition, to remedy, to make up for, to make sound, to renew, to mend, to patch or to otherwise restore function.

The term “replace”, “replacement”, and the like refer to a means to substitute or take the place of defective or injured tissue.

The term “defect” and the like refer to a flaw or a physical problem in a structure, or system, especially one that prevents it from functioning correctly, or a medical abnormality. Defects can include, but are not limited to, wounds, ulcers, burns, natural defects, such as birth defects, and any other defects of biological tissue, including skin, bone, cartilage, muscle, tendon, ligament, arteries and blood vessels, and organs.

The term “injury” and the like refer to wound or trauma; harm or hurt; usually applied to damage inflicted on the body by an external force.

The term “biocompatible” as used in the application means capable of coexistence with living tissues or organisms without causing harm.

The term “extracellular matrix” as used in the application means the substance of a tissue outside and between cells.

The term “moiety” as used in the application means part of a composition that exhibits a particular set of chemical and pharmacologic characteristics. “Biological moieties” are those which derive from living organisms or through protein engineering. “Chemical moieties” do not derive from living organisms.

The term “agent” as used herein means a substance that produces or is capable of producing an effect and would include, but is not limited to, chemicals, pharmaceuticals, biologics, small organic molecules, antibodies, nucleic acids, peptides, and proteins.

The terms “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art, Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

### Carbon Nanotube and Graphene Patch or Implant

Carbon nanotubes (CNTs) are allotropes of carbon with a cylindrical nanostructure (Iijima and Ichihashi (1991)). Nanotubes have been constructed with length-to-diameter ratio of up to 132,000,000:1, significantly larger than for any other material. These carbon nanotubes have unusual properties, which are valuable for nanotechnology, electronics, optics and other fields of materials science and technology. Because of their extraordinary thermal conductivity and mechanical and electrical properties, carbon nanotubes have applications as additives to various structural materials. For instance, nanotubes are part of baseball bats, golf clubs, and car parts (Jorio (2008)).

Carbon nanotubes are the strongest and stiffest materials yet discovered in terms of tensile strength and elastic modulus, respectively. This strength results from the covalent sp2 bonds formed between the individual carbon atoms. In 2000, a multi-walled carbon nanotube was found to have a tensile strength of 63 gigapascals (GPa). Further studies, such as one conducted in 2008, revealed that individual CNT shells have strengths of up to approximately 100 GPa, which is in agreement with quantum/atomistic models. Since carbon nanotubes have a low density for a solid of 1.3 to 1.4 g/cm³, its specific strength of up to 48,000 kN·m·kg⁻¹ is the best of known materials, especially as compared to high-carbon steel with a specific strength of 154 kN·m·kg⁻¹ (Jorio (2008)).

Graphene is a substance made of pure carbon, with atoms arranged in a regular hexagonal pattern similar to graphite, but in a one-atom thick sheet. It is very light, with a 1 square meter sheet weighing only 0.77 milligrams. Graphene is an allotrope of carbon whose structure is a single planar sheet of sp2-bonded carbon atoms, that are densely packed in a honeycomb crystal lattice. Graphene is most easily visualized as an atomic-scale chicken wire made of carbon atoms and their bonds. The crystalline or “flake” form of graphite consists of many graphene sheets stacked together.

The carbon-carbon bond length in graphene is about 0.142 nanometers. Graphene sheets stack to form graphite with an interplanar spacing of 0.335 nanometers. Graphene is the basic structural element of some carbon allotropes including graphite, charcoal, carbon nanotubes and fullerenes. It can also be considered as an indefinitely large aromatic molecule, the limiting case of the family of flat polycyclic aromatic hydrocarbons.

Graphene appears to be one of the strongest materials ever tested. Measurements have shown that graphene has a breaking strength 200 times greater than steel, with a tensile modulus (stiffness) of 1 TPa (150,000,000 psi). Aside from being very strong, graphene is very light, weighing only about 0.77 milligrams per square meter (Skakalova and Kaiser (2014)). The Nobel announcement illustrated this by saying that a 1 square meter graphene hammock would support a 4 kilogram cat but would weigh only as much as one of the cat's whiskers.

It has now been discovered that combining the small size of carbon nanotubes and graphene with their extreme strength provides an implant or patch that will be able to resist biological forces created for example, when sitting and standing. These implants and patches are particularly useful for the repair of annulus fibrosus tissue and to prevent recurrent disk herniation.

The strength of construct is dependent on the amount and size of nanotubes in the construct or patch. The more nanotubes in the construct, the stronger the construct. The less nanotubes and the shorter the nanotubes, the less strong the construct. The flexibility will also depend on the amount and size of nanotubes, with less and shorter nanotubes making the patch more flexible and more nanotubes making the construct less flexible. One of skill in the art will know how to vary the number of rods in order to obtain the optimum strength and flexibility needed for the implant for the particular injury or defect, and the biological tissue being treated, repaired or replaced.

Graphene is found in one-atom thick sheets. The implant or patch of the current invention is made of layers of graphene. The more layers in the patch, the stronger the patch. One of skill in the art will know how to vary the number of layers in order to obtain the strength and flexibility needed for the implant for the particular injury or defect, and the tissue being treated, repaired or replaced.

As set forth below, the implant or patch of the current invention has applicability in the treatment, repair and replacement of a wide variety of biological tissue, ranging from bone to epidermis, As also set forth below, various parameters of strength and flexibility must be considered when formulating the implant.

For applications in repair of tissue defect, the implants need to have modulus similar to the native tissue. As used herein, the term “elastic modulus” refers to an object or substance's tendency to be deformed elastically (i.e., non-permanently) when a force is applied to it. Generally, the elastic modulus of an object is defined as the slope of its stress-strain curve in the elastic deformation region. Specifying how stress and strain are to be measured, including directions, allows for many types of elastic moduli to be defined.

Young's modulus is the mathematical description of an object or substance's tendency to be deformed elastically (i.e., non-permanently) when a force is applied to it, and the object is not confined to any direction perpendicular to the force. Young's modulus describes tensile elasticity or the tendency of an object to deform along an axis when opposing forces are applied along that axis; it is defined as the ratio of tensile stress to tensile strain.

The elastic modulus of an object is defined as the slope of it stress-strain curve in the elastic deformation region. A stiffer material will have a higher elastic modulus.

The bulk modulus (K) describes volumetric elasticity, or the tendency of an object to deform in all directions when uniformly loaded in all directions; it is defined as volumetric stress over volumetric strain, and is the inverse of compressibility, The bulk modulus is an extension of Young's modulus to three dimensions. Three other elastic moduli are Poisson's ratio, Lame's first parameter, and P-wave modulus.

As used herein, the term “shear modulus” refers to the ratio of a measured shear stress to shear strain that is used to produce that stress. The shear modulus or modulus of rigidity (G or μ) describes an object's tendency to shear (the deformation of shape at constant volume) when acted upon by opposing forces. The shear modulus is part of the derivation of viscosity. Generally, the shear modulus can be determined by ASTM test method E143-87 (1998).

As used herein, the term “dynamic modulus” refers to the ratio of stress to strain under vibratory conditions. Dynamic modulus can be calculated from data obtained from either free or forced vibration tests, in shear, compression, or elongation.

While it is within the skill of the art to determine the amount of carbon nanotubes and/or graphene, and carrier material that is needed in the implant for each application set forth below, the amount of carbon nanotubes and graphene to be added would be from about 0.01% by weight up to about 25% by weight in the carrier. For annulus fibrosus and disc repair, the preferred weight ratio of carbon nanotube to carrier is in the range of about 1:10 to about 1:1.5, with about 1:3 being most preferred.

The carrier can be any polymer, plastic, or composite material that is biocompatible. The materials will support the graphene and/or carbon nanotubes, and facilitate the transfer of the nanotubes to the tissue defect. A preferred material is a hydrophilic plastic. This material can include, but is not limited to, polydimethylsiloxane (PDMS), vicryl, polyethylene (PE), poly-lactic acid (PLA), polyetheretherketone (PEEK), polyglycolic acid (PGA), and polyurethane.

PDMS, the most preferred supporting material, belongs to a group of polymeric organosilicon compounds that are commonly referred to as silicones. PDMS is the most widely used silicon-based organic polymer, and is particularly known for its unusual rheological (or flow) properties. PDMS is optically clear, and, in general, inert, non-toxic and non-flammable. It is occasionally called dimethicone and is one of several types of silicone oil (polymerized siloxane). Its applications range from contact lenses and medical devices to elastomers. It is also present in shampoos (as dimethicone, which makes hair shiny and slippery), food (antifoaming agent), caulking, lubricating oils, and heat-resistant tiles.

While the size and thickness of the construct will depend on the tissue, and the injury or defect being treated, repaired or repaired, for annular fibrosus patching, the thickness of the construct should be about 1 to 3 millimeters. As a general rule, the size of the construct should be about 2 to 10 millimeters larger than the injury or defect being repaired, on each side. Again this is possible because the implant is flexible and can be manipulated or folded when inserted or implanted into the defect or injury and then return to its memorialized original shape upon implantation or insertion.

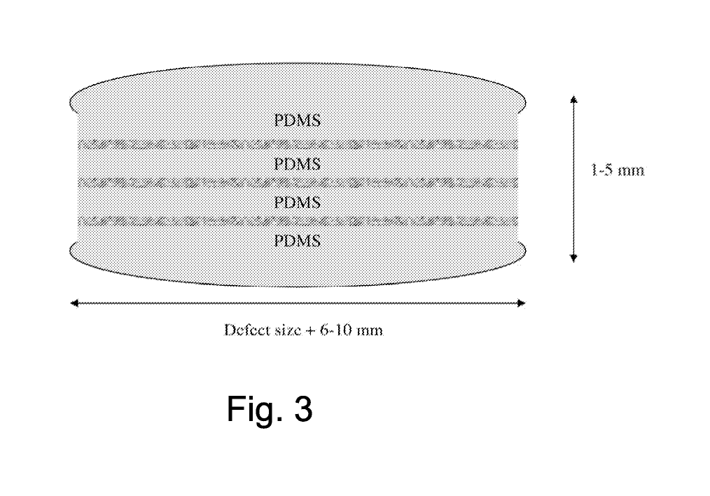

Again while the shape of the implant will be determined by the shape of the defect or injury, the preferred shape for annular fibrosus patches is oval, e.g., a shape similar to a tire. A schematic of this preferred embodiment of the implant or patch used for repair of annulus fibrosus is shown in FIG. 3.

Below is a list of tissues in which the implant can be used, the preferred size and shape, moduli, and the properties of the tissue that must be taken into account when determining the size, strength and flexibility of the implant or patch.

### 1. Bone—

a. Long bone—in cases of bone defect in which an implant is needed, an implant in size of about 1-35 cm in length with a thickness appropriate to the size of the bone used is used. The implant shape will be fabricated according to a patient-specific template of the bone. The implant will have forces that are close to physiological bone properties, i.e., tension about 150 mPa, compression about 200 mPA, shear about 100 mPA, modulus about 17 GPa.

b. Flat bone—in cases of boney defects or cases in which the bone is removed in order to allow room for tissue swelling, the implant size will be anywhere from about 1 cm² to 50 cm². The forces of the implant will provide protection from infections or blunt trauma in the area of the defect but allow swelling of the tissues, i.e, bending, about 3000 N, distraction about 30,000 N.

c. Vertebrae—in cases of removal of vertebrae or fracture of a vertebrae, the shape of the implant will be according to the size removed (changing from cervical, thoracic and lumbar vertebrae). The forces can reach the strength of about 6000 N (load to fail).

2. Cartilage—in cases of focal and large defects of cartilage, a composite will be placed and anchored to the subchondral bone to cover the defects. The modulus will be close to physiological cartilage modulus of about 0.5-0.7 mPA.
3. Meniscus—in cases of resection or damage to the meniscus, a composite shaped as a medial, lateral meniscus or parts of it with tensile strength and modulus about 200 mPa, compressive-aggregate modulus about 0.35-0.45 mPa, and dynamic shear modulus about 0.06-0.09 mPa, is created.
4. Tendon—in case of tendon defects or tears, an implant is created in a specific manner for the tendon that is injured. It can be a flat patch or string shaped. A few examples are:

a. Rotator cuff—about 1-2.0 millimeter thick, 2 cm wide and 5 cm long, failure at about 6000 N, modulus about 830 mPa;

b. Achilles—about 2.0 millimeter thick, 3 cm wide and 8 cm long, failure at about 6000 N, modulus about 830 mPa; and

c. Other tendons—size—about 1-10 cm², force about 1500 N, stiffness about 200 N/mm.

5. Ligaments—in case of ligaments defects or tears, an implant is created in a specific manner for the tendon that is injured. It can be a flat patch or string shaped. A few examples are:

a. ACL and PCL-strength—load about 7500 N, stress about 40 mPa, stiffness about 350 N/mm and linear modulus about 283-345 mPA; and

b. Other ligaments—similar to ACL with load to failure reaching about 10,000 N.

6. Spine disk—in cases of removal of the disk in total or in part in order to preserve motion or to fuse the disk with adjacent levels, a composite is fabricated with compression of about 700-1500 N, and shear about 100-200 N.
7. Joint replacement—in cases of joint replacement, the implant can be used as prosthetic parts for part, or all of the joint. The implant would reach up to compression of about 3000 N and distraction force about 2000 N.
8. Joint interposing—in cases where an interposing graft is placed between two bones or a bone and muscle, ligament or between two organs or spaces in the body.
9. Vessel guard—in order to prevent adhesion between vessels and scar tissue (after a surgery in the vicinity of the vessels or on them).
10. Vessel repair as a graft or implant to vessels.
11. Defects of fascia or compartment in the body natural or surgically created a patch can be placed over the gap to provide closure of the defects.
12. Skin defects—in order to prevent infection and allow proper skin growth.
13. In any void created after the removal of a tumor.
14. A temporary closure method between stages of surgery

Another novel feature of the patch or implant is that it can be made flexible enough to be manipulated or folded when inserted or implanted into the injury or defect, and then open or unfold when in place in the injury or defect upon insertion or implantation. The carrier component of the composite confers “memory” to the material, allowing the device to be folded into a small thin form that will return to its “memorized shape” upon deployment in the defect. Again because the carbon nanotube or graphene portion of the implant is so strong, the result is an implant flexible enough to be folded and have this memory of shape, but also strong enough to withstand the tremendous force applied to many biological tissue, especially musculoskeletal tissue. This is of particular importance when the implant is used for the repair of the annulus fibrosus.

Another novel and most important property of the implant of the current invention is not only is strength combined with small size and flexibility but its lack of immunogeneity. Carbon nanotubes, graphene and PDMS have all been found not to cause immune responses in mammals (see, e.g., Parchi et al. (2013); Popov et al. (2007)).

The patch or implant of the current invention encompasses all carbon and silicon based nanomaterials, known presently or developed in the future.

The implant can have an adhesive added to the external part of the construct. Such adhesives would attach the implant or patch immediately or very soon after insertion, allowing stability of the implant immediately or very soon after insertion, prior to integration with surrounding tissue.

Depending upon the injury or defect, and the site of injury or defect to be treated, repaired and/or replaced, the implant can have adhesive on one side, or a portion or portions, contiguous or non-contiguous, of the external part of the construct, or on the entire surface of the external part of the construct.

Adhesives can work by making the implant or patch hydrophilic or hydrophobic. In particular, the implant made by the exemplified procedure will be hydrophobic, thus, the adhesive makes the implant hydrophilic. Moreover, adhesives can work by binding to collagen or another component of the extracellular matrix. In particular, amine groups (NH₃) are very active in binding to collagen. Thus, compositions that contain amine groups are useful as adhesives. Such adhesives would include but are not limited to PEG and poly-L-lysine.

These adhesives can be attached to the implants by any method known in the art. In particular, the implant can be treated with oxygen plasma and dipped in poly-L-lysine.

One particular preferred adhesive are those disclosed in commonly-owned U.S. Pat. No. 8,440,618, which is incorporated by reference in its entirety. Such adhesives are chemical and biological moieties having the ability to bind to a component of the extracellular matrix of the host tissue upon implantation. Upon implantation, the moiety of the composition would allow the implant to integrate with the extra-cellular matrix components of the host tissue in a short period of time. In a preferred embodiment, the moiety would bond with collagen, thus, any tissue that contains collagen in its extracellular matrix is a candidate for implantation of the composition.

In a preferred embodiment, the moiety is chemical, and in a most preferred embodiment, contains a chemically reactive group, such as a carbonate (“open carbonate” or “OC”).

In another preferred embodiment, the moiety is biological. Biological moieties would be derived from living organisms or through protein engineering, and could include, but are not limited to, proteins, protein sub-domains, and mutated proteins with altered affinity for a ligand, in particular, collagen. One source for biological moieties would be bacteria, including but not limited to Staphylococcus aureus, Enterococcus faecalis, and Streptococcus mutans. Other sources would be mammalian collagen binding proteins, such as decorin. A preferred biological moiety is a protein derived from Staphylococcus aureus, encoded by the collagen adhesion gene, CNA.

Other agents can be optionally added to the implant or patch, either externally or internally. Any agent that facilitates migration, integration, regeneration, proliferation, and growth of cells into and around the implant or patch composition, and/or the injury or defect, and/or promotes healing of the injury or defect, and/or are chondrogenic and osteogenic, i.e., build bone and cartilage, can be added to the implant or patch.

These agents include, but are not limited to, cytokines, chemokines, chemoattractants, anti-microbials, anti-virals, anti-inflammatories, pro-inflammatories, bone or cartilage regenerator molecules, and chemotherapy medication, as well as combinations thereof, specific for the injury or defect being treated, repaired, and/or replaced.

Cytokines for use in the invention include, but are not limited to, interleukins (e.g., IL-13), interferons, transforming growth factor (TGF), epidermal growth factor (EGF), insulin growth factor (IGF), fibroblast growth factor (FGF), vascular endothelial growth factor (VEGF), dermal growth factor, stem cell factor (SCF), granulocyte-colony stimulating factor (G-CSF), granulocyte-macrophage stimulating factor (GM-CSF), stromal cell-derived factor-1, steel factor, platelet derived growth factor (PDGF), angiopoeitins (Ang), hepatocyte growth factor, insulin-like growth factor (IGF-1), colony-stimulating factors, thrombopoietin, erythropoietin, fit3-ligand, and tumor necrosis factor α (TNFα) as well as combinations thereof.

Chemokines include, but are not limited to, CC, CXC, C, and CX₃C chemokines.

Chemoattractants include, but are not limited to, bone morphogenic protein (BMP).

These chemokines, cytokines, and chemoattractants will have the ability to stimulate cell migration, proliferation, and regeneration around and into the defect or injury, as well as promote adhesion, and synthesis of the extracellular matrix.

Anti-microbial agents include, but are not limited to, 3-lactam antibiotics, such as cefoxitin, n-formamidoyl thienamycin and other thienamycin derivatives, tetracyclines, chloramphenicol, neomycin, gramicidin, bacitracin, sulfonamides, aminoglycoside antibiotics such as gentamycin, kanamycin, amikacin, sisomicin and tobramycin, nalidixic acids and analogs such as norfloxican, the antimicrobial combination of fluoroalanine/pentizidone, and nitrofurazones.

Anti-inflammatory agents are agents that inhibit or prevent an immune response in vivo. Exemplary anti-inflammatory agents include: agents which inhibit leukocyte migration into the area of injury (“leukocyte migration preventing agents”); and antihistamines. Representative leukocyte migration preventing agents include, but are not limited to, silver sulfadiazine, acetylsalicylic acid, indomethacin, and Nafazatrom. Representative anti-histamines include, but are not limited to, pyrilamine, chlorpheniramine, tetrahydrozoline, antazoline, and other anti-inflammatories such as cortisone, hydrocortisone, beta-methasone, dexamethasone, fluocortolone, prednisolone, triamcinolone, indomethacin, sulindac, its salts and its corresponding sulfide.

Pro-inflammatory agents would be added to an implant or patch when the generation of scar tissue is desired to increase the stability of the implant or patch, such as when the implant or patch is being implanted into a fascia defect or the annulus to allow healing of scar tissue in a controlled manner.

Additional agents that can be included or added to the patch or implant could include, for example: aminoxyls, furoxans, nitrosothiols, nitrates and anthocyanins; nucleosides, such as adenosine; and nucleotides, such as adenosine diphosphate (ADP) and adenosine triphosphate (ATP); neurotransmitter/neuromodulators, such as acetylcholine and 5-hydroxytryptamine (serotonin/5-HT); histamine and catecholamines, such as adrenalin and noradrenalin; lipid molecules, such as sphingosine-1-phosphate and lysophosphatidic acid; amino acids, such as arginine and lysine; peptides such as the bradykinins, substance P and calcium gene-related peptide (CGRP), and proteins, such as insulin, vascular endothelial growth factor (VEGF), and thrombin.

Other agents can include pharmaceutically active compounds, hormones, enzymes, DNA, plasmid DNA, RNA, siRNA, viruses, proteins, lipids, pro-inflammatory molecules, antibodies, anti-sense nucleotides, and transforming nucleic acids or combinations thereof.

### Method of Manufacturing the Carbon Nanotube and Graphene Patch or Implant

A further embodiment of the present invention is a novel method for manufacturing or producing patches or implants made from carbon nanotubes and/or graphene.

This method utilizes the extremely small and strong material of carbon nanotubes or graphene and combines them with a supporting material. This supporting material also facilitates the transfer of the carbon nanotubes or graphene into the defect.

As discussed above, the supporting material can include any biocompatible polymer, plastic or composite material with hydrophobic plastic being preferred This material can include, but is not limited to, polydimethylsiloxane (PDMS), vicryl, polyethylene (PE), poly-lactic acid (PLA), polyetheretherketone (PEEK), polyglycolic acid (PGA), and polyurethane.

A preferred supporting material is PDMS.

In preparing the carbon nanotube patch, solid carbon nanotubes are put into solution. The preferred weight of the carbon nanotubes is one (1) milligram.

The carbon nanotubes are dissolved in a solvent. Any solvent know in the art can be used. A preferred solvent is dichloroethane.

The carbon nanotubes are dissolved by any method known in the art that ensures a uniform dispersion of the nanotubes. A preferred method is sonication.

The resulting carbon nanotube solution is mixed with the supporting material or carrier. The range of carbon nanotubes to be added is from about 0.01% to about 25% by weight in the carrier. For patches for annulus fibrosus and disc repair, the preferred weight ratio of carbon nanotube to carrier is in the range of about 1:10 to about 1:1.5, with about 1:3 being most preferred.

A hardener can be added.

The solution is mixed and can be kept under vacuum as well.

The film is then hardened by heat, or any other method known in the art.

It is preferred that the film is hardened on a hot plate for twenty (20) minutes at 60° C. However, one of skill in the art will realize that the temperature and time will depend on the material being hardened.

The preparation of the patch comprising graphene starts with the preparation of large scale graphene. A preferred method for this is the CVD copper growth method.

The graphene on copper is then laminated onto the supporting material, which is preferably PDMS is the form of a thin film. The copper is etched with a solution of ammonium persulfate.

The copper is then removed. The supporting material with an optional hardener is transferred onto the graphene by spincoating or another method known in the art.

The supporting material is hardened. The preferred method for hardening is heating to 60° C. However, one of skill in the art will realize that the temperature and time will depend on the material being hardened.

The range of graphene to be added is from about 0.01% to about 25% by weight in the carrier.

This process is repeated with another graphene on copper foil being laminated on the film. The process can be repeated from about 2 to 10 times with the preferred number being 4 to 5 times.

Implants or patches comprising both carbon nanotubes and graphene can also be manufactured by the method of the current invention, by combining the methods set forth above.

Any adhesives would be added by physical application. The surface chemistry is performed by first activating the surface of the carrier film and then using attachment chemistry. One example would be the oxidation of PDMS with an oxygen plasma, and then using silane chemistry to attach specific agents. This method also allows the attachment of other agents such as chemokines and anti-inflammatories.

Adhesives and other agents can be attached to the implant or patch using linkers.

### Method of Uses of the Carbon Nanotube and Graphene Implants and Patches

The implant or patch of the current invention can be used in a method to treat, repair and/or replace various defects and/or injuries in biological tissues, in a subject in need thereof, most preferably a mammal, and most preferably a human.

Because of the many novel and beneficial qualities of the patch of the current invention, including extreme strength in a small size, its memory of shape, biocompatibility, and lack of immunogenicity, there are several applications of use of the implant or patch in treating, replacing and/or repairing biological tissue.

In a preferred embodiment, the tissue is musculoskeletal tissue. Musculoskeletal tissue contemplated to be treated, replaced and/or repaired by the method includes bone (long, flat, and vertebrae), tendon, ligaments, cartilage, meniscus, and the discs of the spine. The most preferred embodiment is a method to repair the annular fibrosus tissue in the discs of the spine.

Other preferred uses would be as a replacement or repair of long bones, such as femurs, or flat bones, in particular the skull. It is contemplated that the patch or implant of the current invention can be used to replace parts of the skull that have been lost in trauma or disease.

The implants or patches can also be used to treat, replace and/or repair fascia tissue such as in hernia repair and other injuries or defects of fascia tissue.

The implants can also be used with silicon implants and as an interposition to joints (such as the thumb carpo or meta carpal joint or the temporal mandibular joint).

The implants or patches of the current invention can also be used to treat, replace and/or repair dura tissue, in particular patching dura tissue that has been lost to trauma or disease.

Additionally the implants or patches can be used for treatment, repair, and/or replacement of the aorta, and other arteries and vessels.

The implants or patches of the current invention can also be used for treatment, repair and/or replacement of various organs throughout a mammal, such as the heart, lungs, stomach, liver, kidneys, and intestines.

The patch can also be used for wound repair of the epidermis tissue.

When used for treatment, repair and/or replacement of organs and epidermis tissue, it is contemplated that the patch composition would include additional agents to assist in healing such as anti-inflammatory agents, and anti-infectious agents. One such agent would be silver. Also when used for these applications, especially in epidermis tissue, it is contemplated that the patch could be removed after the tissue has repaired and regenerated itself.

As stated, because of its novel and highly beneficial properties, the patch of the current invention is well suited for treatment, repair or replacement of biological tissue, however, these properties also make the patch useful for other applications.

The patch can be used as a marker after surgery to assist in locating the area of the surgery. It can also be used as a barrier after surgery. The barrier can prevent blood vessels and other biological tissue from attaching to scar tissue. The barrier can also be used as protection of biological tissue in further repeat surgeries.

It is contemplated that when the implant or patch is used for these applications, they can be removed from the biological tissue if necessary.

Patches or implants that further contain adhesive, such as an agent that renders the patch or implant hydrophilic or hydrophobic, or binds to a component or the extracellular matrix, or a biological or chemical moiety that binds to a component of the extracellular matrix, can also be used to treat, replace and/or repair various tissues in a subject in need thereof, most preferably a mammal, and most preferably a human. In a preferred embodiment, the tissue would contain collagen in the extracellular matrix, and most preferably musculoskeletal tissue, and most preferably, the annular fibrosus tissue in the discs of the spine. Patches or implants with this adhesive attach immediately to the host tissue, and provide increased interfacial strength to the implant immediately upon implantation.

Patches or implants that further contain chemokines, cytokines, and/or chemoattractants, as well as other agents, allow for the regeneration, proliferation and migration of cells to the implantation site allowing of continued increase in strength.

Those of skill in the art would appreciate that method of treating, replacing and/or repairing various tissues with the carbon nanotube and/or graphene patches may include operative techniques and procedures, utilizing techniques such as magnetic resonance and computer guided technology.

In one preferred embodiment, the method is for treating, replacing and/or repairing defects of annulus fibrosus tissue of the spine. In this embodiment, methods known in the art for approaching the disc including but not limited to traditional disketomy, can be used. A slit is opened in the defect using any method known in the art, including but not limited to, the use of forceps or other surgical tools, such as knives. The patch is then inserted into the slit, using a tool specially designed for this use, or another surgical tool such as forceps. It is preferred that the patch or implant be manipulated or folded upon insertion, and then open or unfold after insertion. The disc can be approached from the anterior, the posterior or the side.

In this novel method, the device is placed on the inner side of the defect, similar to a bicycle tire patch, preventing bulge-out of the nucleus pulposus and recurrence of disc herniation. The carrier component of the composite confers “memory” to the material, allowing the device to be folded into a small form that will return to its memorized shape upon deployment in the defect, i.e., once placed on the inner side of the annulus. In this manner, an implant or patch that is large enough and strong enough to repair the defect or injury can be inserted into the inner side of annulus without highly invasive techniques.

The method also encompasses the determination of the size and shape of the carbon nanotube or graphene patch using techniques known in the art. The patch should be about 2 to 10 mm larger than the defect, and the shape determined by the shape of the defect.

Additionally, carbon nanotubes and graphene could be added to various orthopedic materials that are FDA approved, including but not limited to PE (polyethylene), PLA (poly-lactic acid), PEEK (polyetheretherketone), PGA (polyglycolic acid), polyurethane, and PMMA (acrylic cement). For the latter, a compression modulus of about 100 mPA is necessary. The nanotubes would increase stiffness and improve osteointegression. All of these materials have wear and strength limitations that might be improved with nanotubes.

A further orthopedic area for use of the carbon nantubes or graphene implants is that of bone cements. Bone cements generally set up and harden like spackle in drywall.

However, new techniques use a drillable version similar to a screw through the cement. Carbon nanotubes or graphene would help to stabilize and strengthen these cements.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Preparation of an Implant Comprising Carbon Nanotubes

One (1) milligram of arc-discharged carbon nanotubes (Hanwha Nanotech) was dissolved in ten (10) milliliters of dichloroethane and sonicated in a bath sonicator for four (4) hours to ensure uniform dispersion of the carbon nanotubes. The solution was then centrifuged at 1000 rpm for one (1) minute to obtain a clear solution of carbon nanotubes,

The resulting carbon nanotube solution was mixed with PDMS in a 1:3 weight ratio and a PDMS hardener was added. The solution was mixed, and when thoroughly mixed was kept under vacuum for one (1) hour to remove air bubbles in solution.

The resulting mixture was poured into a substrate to form a thin film. The film was hardened on a hot plate at 60° C. for twenty (20) minutes.

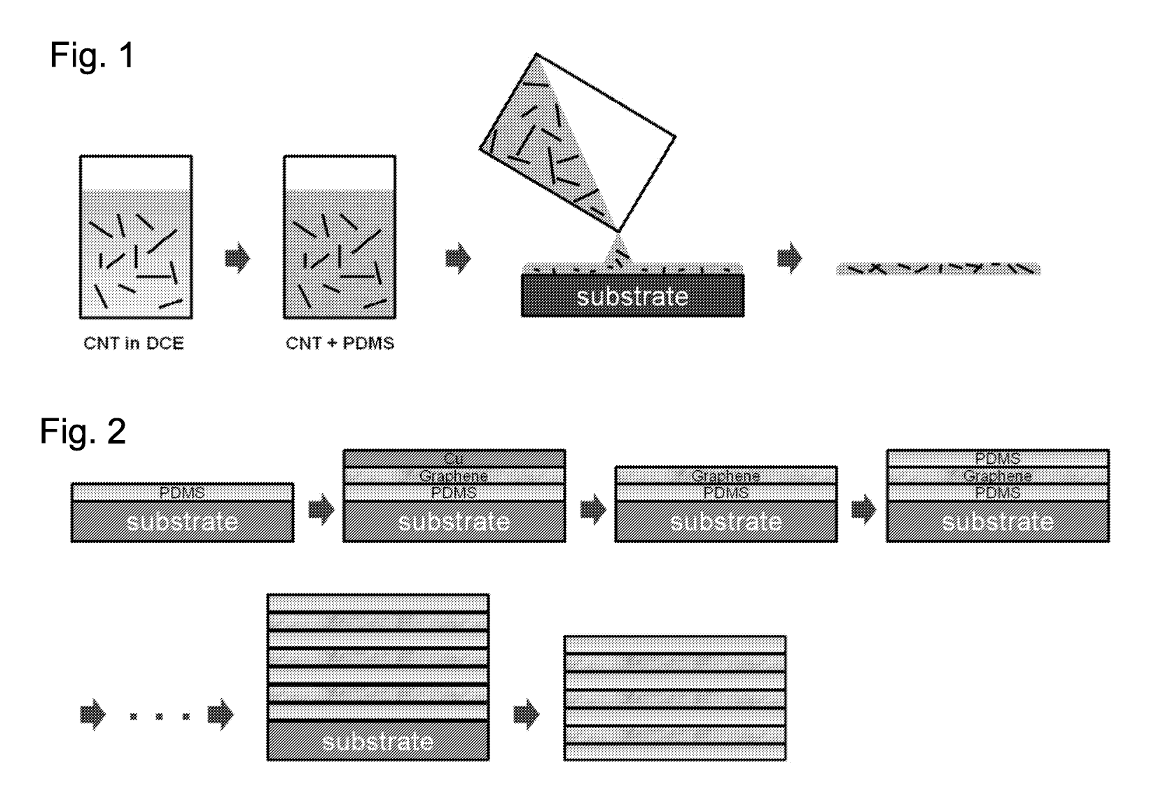

A schematic of this process and the resulting patch is shown in FIG. 1.

### Example 2—Further Preparation of Implants Comprising Carbon Nanotubes

Using the procedure of Example 1, implants were made using carbon nanotubes where the carbon nanotube solution was mixed with the PDMS in weight ratios ranging from 1:1.5 to 1:10, all with satisfactory results, illustrating that a range of weight ratios of carbon nanotubes to carrier can be used in the manufacture of the implants.

### Example 3—Preparation of an Implant Comprising Graphene

A large-scale graphene on copper foil was grown by the CVD growth method. Graphene on copper was laminated onto a thin PDMS film and copper was etched with ammonium persulfate solution.

After the copper was fully removed, the sample was gently rinsed with water and air dried.

PDMS which was pre-mixed with a hardener, was spincoated onto the graphene transferred PDMS film. The PDMS was baked at 60° C. to harden.

Another graphene on copper foil was laminated onto the film.

This process was repeated four or five times, to create a multilayer graphene patch.

A schematic of this process and the resulting patch is shown in FIG. 2.

### Example 4—Insertion of the Carbon Nanotube Patch

A cadaver lumbar spine was defrosted and a 5 mm by 5 mm defect was created in the anterior aspect of the disc, penetrating the annulus. Using forceps, the slit was opened and implant manufactured as set forth in Example 1, was inserted after being folded into the forcep. The size of the implant was larger than the defect, reaching a size of close to 1 cm by 1 cm. After insertion, axial and rotational forces were applied to the spine. The implant stayed in place and did not fold or tear.

The patch was retrieved after the forces were applied and testing was performed that verified that the patch sustained the forces.

### Example 5—Insertion of the Graphene Patch

A cadaver lumbar spine was defrosted and a 5 mm by 5 mm defect was created in the anterior aspect of the disc, penetrating the annulus. Using forceps, the slit was opened and implant manufactured as set forth in Example 3, was inserted after being folded into the forcep. The size of the implant was larger than the defect, reaching a size of close to 1 cm by 1 cm. after insertion, axial and rotational forces were applied to the spine. The implant stayed in place and did not fold or tear.

The patch was retrieved after the forces were applied and testing was performed that verified that the patch sustained the forces.

### Example 5—Attachment of Adhesive to the Implant

The implant was treated with oxygen plasma (250 mTorr) for five (5) minutes to make the surface hydrophilic. The implant was then dipped in a poly-L-lysine solution (0.1 wt %) for 10 minutes to obtain self-assembled layer of poly-L-lysine. The implant was gently rinsed and blow-dried with argon gas.

### REFERENCES

Carragee et al. (2003) J. Bone Joint Surg. Am. 85:102

Christenson et al. (2007) J. Orthop. Research 25:11

Iijima and Ichihashi (1991) Nature 354:56

Jorio (2008) Carbon Nanotubes: Advanced Topics in the Synthesis, Structure, Properties and Applications (Topics in Applied Physics) Dresselhaus and Dresslhaus Ed., Springer, Volume 111

Nerururkar et al.(2009) Nat. Mater. 8:986;

Parchi et al. (2013) J. Nanomed. Biother. Discov. 3:2;

Popov et al. (2007) Int'l J. Nanomed. 2:361

Skakalova and Kaiser Ed. (2014) Graphene: Properties, Preparation, Characterization and Devices Woodhead Publishing

Spangfort et al. (1972) Acta Orthopaedica Scandinavia Supplement 142

1. A composition for the treatment, repair or replacement of biological tissue, comprising carbon nanotubes and a supporting material comprising a biocompatible plastic.

2. (canceled)

3. The composition of claim 1, wherein the supporting material is selected from the group consisting of polyethylene glycol (PEG), polydimethylsiloxane (PDMS), vicryl, polyethylene (PE), poly-lactic acid (PLA), polyetheretherketone (PEEK), polyglycolic acid (PGA), and polyurethane.

4. The composition of claim 1, wherein the composition further comprises an adhesive.

5-17. (canceled)

18. The composition of claim 1, wherein the biological tissue is selected from the group consisting of musculoskeletal, fascia, dermis, epidermis, blood vessels or arteries, aorta and organs.

19-21. (canceled)

22. The composition of claim 1, wherein the composition can be manipulated or folded into a smaller size and/or different shape, and upon insertion or implantation into the biological tissue, opens or unfolds into its original size and/or shape.

23. A composition for the treatment, repair or replacement of biological tissue, comprising graphene and a supporting material comprising a biocompatible plastic.

24. (canceled)

25. The composition of claim 23, wherein the supporting material is selected from the group consisting of polyethylene glycol (PEG), polydimethylsiloxane (PDMS), vicryl, polyethylene (PE), poly-lactic acid (PLA), polyetheretherketone (PEEK), polyglycolic acid (PGA), and polyurethane.

26. The composition of claim 23, wherein the composition further comprises an adhesive.

27-39. (canceled)

40. The composition of claim 23, wherein the biological tissue is selected from the group consisting of musculoskeletal, fascia, dermis, epidermis, blood vessels or arteries, aorta and organs.

41-43. (canceled)

44. The composition of claim 23, wherein the composition can be manipulated or folded into a smaller size and/or different shape, and upon insertion or implantation into the biological tissue, opens or unfolds into its original size and/or shape.

45. A composition for the treatment, repair or replacement of biological tissue, comprising carbon nanotubes, graphene and a supporting material.

46. A method of manufacturing the composition of claim 1, comprising:
a. preparing a solution of carbon nanotubes;
b. mixing the carbon nanotube solution with a supporting material;
c. pouring the mixture of carbon nanotube solution and supporting material onto a substrate to form a thin film; and
d. hardening the film.

47. (canceled)

48. The method of claim 46, wherein the supporting material is selected from the group consisting of polyethylene glycol (PEG), polydimethylsiloxane (PDMS), vicryl, polyethylene (PE), poly-lactic acid (PLA), polyetheretherketone (PEEK), polyglycolic acid (PGA), and polyurethane.

49. (canceled)

50. A method for manufacturing the composition of claim 23, comprising:
a. preparing a large scale graphene on copper foil;
b. laminating the graphene on copper onto a supporting material to make a film;
c. removing the copper,
d. adding additional supporting material to the film; and
e. hardening the film.

51. (canceled)

52. The method of claim 50, wherein the supporting material is selected from the group consisting of polyethylene glycol (PEG), polydimethylsiloxane (PDMS), vicryl, polyethylene (PE), poly-lactic acid (PLA), polyetheretherketone (PEEK), polyglycolic acid (PGA), and polyurethane.

53. (canceled)

54. A method of treating, repairing or replacing a defect or injury in biological tissue comprising inserting the composition of claim 1 into the biological tissue of a subject in need thereof.

55. The method of claim 54, wherein the biological tissue is selected from the group consisting of musculoskeletal, fascia, dermis, epidermis, blood vessels or arteries, aorta and organs.

56-60. (canceled)

61. A method of treating, repairing or replacing a defect or injury in biological tissue comprising inserting the composition of claim 23 into the biological tissue of a subject in need thereof.

62. The method of claim 61, wherein the biological tissue is selected from the group consisting of musculoskeletal, fascia, dermis, epidermis, blood vessels or arteries, aorta and organs.

63-67. (canceled)

68. A method of treating, repairing or replacing a defect or injury in the annular fibrosus tissue of the discs of the spine in a subject in need thereof, comprising:
a. creating an opening in or near the defect or injury;
b. manipulating the composition of claim 1 to a smaller size and/or different shape;
c. inserting the composition into the opening and into the inner side of the annular fibrosus; and
d. allowing the composition to return to its original size and/or shape, wherein the composition before manipulation is larger than the injury or defect.

69-70. (canceled)

71. A method of treating, repairing or replacing a defect or injury in the annular fibrosus tissue of the discs of the spine in a subject in need thereof, comprising:
a. creating an opening in or near the defect or injury;
b. manipulating the composition of claim 23 to a smaller size and/or different shape;
c. inserting the composition into the opening and into the inner side of the annular fibrosus; and
d. allowing the composition to return to its original size and/or shape, wherein the composition before manipulation is larger than the injury or defect.

72-73. (canceled)
